(19) United States
(12) Patent Application Publication
(10) Pub. No.: US 2021/0379336 A1
(43) Pub. Date: Dec. 9, 2021

(54) Catheter with Variable Length Extension Leg

(71) Applicant: Bard Access Systems, Inc., Salt Lake City, UT (US)

(72) Inventor: Kyle G. Thornley, Farmington, UT (US)

(21) Appl. No.: 17/342,285

(22) Filed: Jun. 8, 2021

Related U.S. Application Data
(60) Provisional application No. 63/036,899, filed on Jun. 9, 2020.

(51) Int. Cl. A61M 25/02; A61M 25/00

(52) U.S. Cl. CPC A61M 25/02; A61M 25/002; A61M 25/0014; A61M 25/0097

(57) ABSTRACT

A catheter assembly for organizing a catheter system is disclosed. The catheter assembly can include a molded hub, a catheter tube coupled to a distal end of the molded hub, a plurality of extension legs having distal ends coupled to the molded hub, and a securing mechanism. The molded hub can include a pair of hub lumens forming a hub portion of a pair of fluid pathways through the catheter assembly. The catheter tube can include a pair of catheter tube lumens. The securing mechanism can be configured to secure the plurality of extension legs together. The securing mechanism can be in the form of a slidable collar including a proximal opening and distal opening, wherein the slidable collar is positioned over the plurality of extension legs.

### PRIORITY

This application claims the benefit of priority to U.S. Provisional Application No. 63/036,899, filed Jun. 9, 2020, which is incorporated by reference in its entirety into this application.

### BACKGROUND

In current catheters, extension legs are only secured at the hub and are free to move proximal of the hub. Extension legs contain clamps and Luer locks often causing them to be bulky and hard to secure together. This can frustrate medical professionals and patients that need to perform dressing changes at the site of the catheter and can lead to placing an inadequate occlusive seal at the catheter site. There currently exists no solution for helping medical professionals secure the extension legs together proximally from the hub so that they may focus on the dressing change.

Disclosed herein are a catheter assembly for organizing and securing the plurality of extension legs and a method of making the catheter assembly for organizing and securing the plurality of extension legs.

### SUMMARY

Disclosed herein are a catheter assembly system and methods of use and manufacturing the same. The disclosed embodiments of a catheter assembly system provide several advantages over conventional catheter assembly systems. Particularly, embodiments of the disclosure enable medical professions to easily secure a plurality of extension legs of the catheter assembly system. Such is beneficial while attempting to create an occlusive seal at the catheter entry site and during general use as such provides comfort and convenience for the patient.

In some embodiments, a catheter assembly for organizing a catheter system is shown comprising a molded hub including at least a pair of hub lumens, a catheter tube coupled to a distal end of the molded hub, the catheter tube including at least a pair of catheter tube lumens, a plurality of extension legs, each including a distal end coupled to a proximal end of the molded hub, wherein a fluid pathway is formed between the plurality of extension legs and the catheter tube through the pair of hub lumens, and a securing mechanism configured to secure the plurality of extension legs together, wherein the securing mechanism is a slidable collar positioned over the plurality of extension legs.

In some embodiments, the slidable collar is a permanent slidable collar comprising a length, a width and a height, having a proximal end with a proximal opening, a distal end with a distal opening, with an outer surface and an inner surface.

In some embodiments, the length is greater than or equal to the width, and the width is greater than or equal to the height. In some embodiments, the outer surface slopes towards the inner surface at a longitudinal midpoint, and slopes away from the inner surface towards a longitudinal endpoint. In some embodiments, the inner surface is configured to create friction with an exterior of the extension legs, wherein the friction resists movement of the slidable collar in an absence of an external force applied to the slidable collar. In some embodiments, the proximal opening and the distal opening have a shape configuration of an oval.

In some embodiments, a catheter assembly for organizing an catheter system comprising a molded hub including at least a pair of hub lumens, the hub lumens forming a hub portion of a pair of fluid pathways through the catheter assembly, a catheter tube coupled to a distal end of the molded hub, the catheter tube including at least a pair of catheter tube lumens, a plurality of extension legs each including a proximal and a distal end, wherein the distal end of each extension leg is coupled to the molded hub, and a securing mechanism configured to secure the plurality of extension legs together, wherein the securing mechanism includes one or more of an adhesion bond between the plurality of extension legs, or an attachable slidable collar or a permanent slidable collar. The securing mechanism may include the adhesion bond between the plurality of extension legs. In some embodiments, the securing mechanism is the attachable slidable collar.

In some embodiments, the attachable slidable collar is detachably coupled to the molded hub. In some embodiments, the attachable slidable collar or permanent slidable is positioned over the plurality of extension legs. In some embodiments, the securing mechanism is either of the attachable slidable collar or the permanent slidable collar, and wherein the securing mechanism comprises a length, a width, a height, having a proximal end with a proximal opening, a distal end with a distal opening, a longitudinal midpoint, a longitudinal endpoint and with an outer surface and an inner surface.

In some embodiments, the length is greater than or equal to the width, and the width is greater than or equal to the height. In some embodiments, the outer surface slopes towards the inner surface at the longitudinal midpoint, then slopes away from the inner surface towards the longitudinal endpoint. In some embodiments, the inner surface is configured to create friction with an exterior of the extension legs, wherein the friction resists movement of the attachable slidable collar in an absence of an external force applied to the attachable slidable collar. In some embodiments, the proximal opening and the distal opening are oval shaped.

In some embodiments, the securing mechanism can be separated axially into two separate sections. In some embodiments, the securing mechanism is joined at the longitudinal midpoints. In some embodiments, the securing mechanism is joined together by a first hinge and a first snap, opposite the first hinge. In some embodiments, the securing mechanism is joined together by a first clasp and a second clasp, opposite the first clasp. In some embodiments, the securing mechanism is joined together by a first pair of magnets and a second pair of magnets, opposite the first pair of magnets.

In some embodiments, a method of making a catheter assembly for organizing a catheter system is disclosed. The method may comprise operations including obtaining a molded hub including at least a pair of hub lumens, each of the hub lumens having an entry point adjacent or substantially adjacent to another entry point, obtaining a catheter tube, the catheter tube including at least a pair of catheter tube lumens, obtaining a plurality of extension legs, obtaining a securing mechanism for securing the plurality of extension legs together, wherein the securing mechanism is selected from the group consisting of an adhesion bond, a permanent slidable collar, and an attachable slidable collar, coupling the catheter tube to a distal end of the molded hub, coupling a distal end of each of the plurality of extension legs to a proximal end the molded hub, wherein a fluid pathway is formed between the plurality of extension legs and the catheter tube through the pair of hub lumens, securing the plurality of extension legs together using the securing mechanism, and packaging the catheter assembly into a single use packaging.

In some embodiments, the securing the plurality of extension legs together using the securing mechanism further comprises curing the plurality of extension legs together with heat to create the adhesion bond. In some embodiments, the securing the plurality of extension legs together using the securing mechanism further comprises coating the plurality of extension legs with an adhesive and curing with heat, pressure or a combination thereof to create the adhesion bond.

In some embodiments, the securing the plurality of extension legs using the securing mechanism further comprises attaching the permanent slidable collar. In some embodiments, the securing the plurality of extension legs using the securing mechanism further comprises attaching the attachable slidable collar.

These and other features of the concepts provided herein will become more apparent to those of skill in the art in view of the accompanying drawings and following description, which describe particular embodiments of such concepts in greater detail.

### BRIEF DESCRIPTION OF THE DRAWINGS

Embodiments of the invention are illustrated by way of example and not by way of limitation in the figures of the accompanying drawings, in which like references indicate similar elements and in which:

FIG. 1 illustrates an exemplary view of a catheter assembly in accordance with some embodiments;

FIGS. 2A-2B illustrate exemplary views of a catheter assembly for organizing a catheter system including a securing mechanism further including an adhesive bond in accordance with some embodiments;

FIGS. 3A-3B illustrate exemplary views of a catheter assembly for organizing a catheter system including a securing mechanism further including a permanent slidable collar in accordance with some embodiments;

FIGS. 4A-4B illustrate exemplary views of a catheter assembly for organizing catheter system including a securing mechanism further including an attachable slidable collar in accordance with some embodiments;

FIGS. 5A-5B illustrate a method of use for organizing a plurality of extension legs in accordance with some embodiments;

FIGS. 6A-6B illustrate alternative embodiments of slidable collars in accordance with some embodiments;

FIG. 7 illustrates a flowchart illustrating a method for making a catheter assembly for organizing a catheter system in accordance with some embodiments;

FIG. 8 is an illustration of a catheter assembly including a slidable collar secured to a patient using a dressing in accordance with some embodiments; and

FIGS. 9A-9B are perspective views of an anchoring system in accordance with some embodiments.

### DETAILED DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise.

With respect to “proximal,” a “proximal portion” or a “proximal-end portion” of, for example, a catheter disclosed herein includes a portion of the catheter intended to be near a clinician when the catheter is used on a patient. Likewise, a “proximal length” of, for example, the catheter includes a length of the catheter intended to be near the clinician when the catheter is used on the patient. A “proximal end” of, for example, the catheter includes an end of the catheter intended to be near the clinician when the catheter is used on the patient. The proximal portion, the proximal-end portion, or the proximal length of the catheter can include the proximal end of the catheter; however, the proximal portion, the proximal-end portion, or the proximal length of the catheter need not include the proximal end of the catheter. That is, unless context suggests otherwise, the proximal portion, the proximal-end portion, or the proximal length of the catheter is not a terminal portion or terminal length of the catheter.

With respect to “distal,” a “distal portion” or a “distal-end portion” of, for example, a catheter disclosed herein includes a portion of the catheter intended to be near or in a patient when the catheter is used on the patient. Likewise, a “distal length” of, for example, the catheter includes a length of the catheter intended to be near or in the patient when the catheter is used on the patient. A “distal end” of, for example, the catheter includes an end of the catheter intended to be near or in the patient when the catheter is used on the patient. The distal portion, the distal-end portion, or the distal length of the catheter can include the distal end of the catheter; however, the distal portion, the distal-end portion, or the distal length of the catheter need not include the distal end of the catheter. That is, unless context suggests otherwise, the distal portion, the distal-end portion, or the distal length of the catheter is not a terminal portion or terminal length of the catheter.

Unless defined otherwise, all technical and scientific terms used herein have the same meaning as commonly understood by those of ordinary skill in the art.

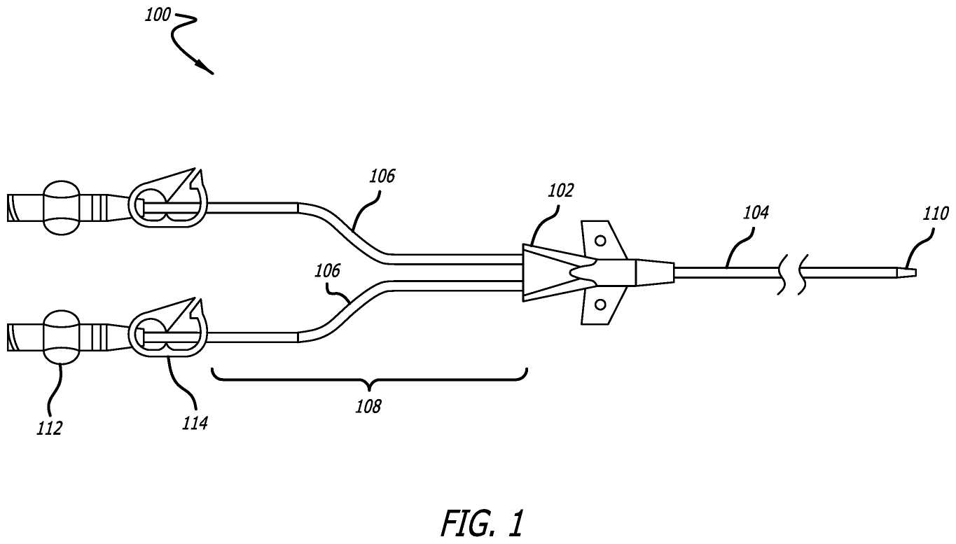

FIG. 1 shows a molded hub 102 that receives a plurality of extension legs 106, clamps 114 and Luer connectors 112, with the clamps 114 and the Luer connectors 112 connected to the plurality of extension legs 106. A catheter tube 104 containing a catheter tip 110 is coupled to the molded hub 102. The plurality of extension legs 106 are not connected together along the length 108 of the plurality of extension legs 106, which allows the clamps 114 and Luer connectors 112 the ability to move laterally along each of the plurality of extension legs 106. When performing a dressing change, medical professionals often need to secure the plurality of extension legs 106, clamps 114 and Luer connectors 112 proximal the catheter entry site, while attempting to create an occlusive seal at the catheter entry site. Creating an occlusive seal is important in maintaining sterility and ensuring the catheter entry site does not become infected. Organizing and securing the plurality of extension legs 106, clamps 114 and Luer connectors 112 can also increase comfort for the patient.

### Catheter Assembly with Adhesion Bond

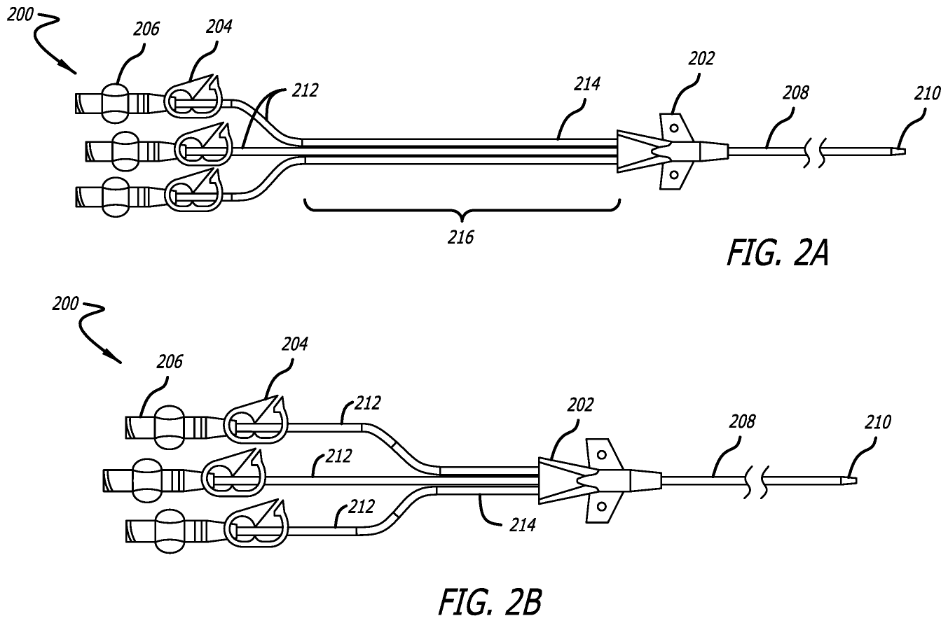

FIG. 2A illustrates an exemplary view of a catheter assembly for organizing a catheter system 200 having extension legs coupled via an adhesion binding. In FIG. 2A, a molded hub 202 is coupled to a catheter tube 208 including a catheter tip 210. A plurality of extension legs 212, having disposed thereon clamps 204 and Luer connectors 206, are connected to the molded hub 202. A securing mechanism 214, e.g., the adhesion binding, secures and organizes the plurality of extension legs 106 along the length of the plurality of extension legs 216 proximal the molded hub 102.

The molded hub 202 may include at least a pair of hub lumens, where one example includes an arterial lumen and a venous lumen. However, the embodiment is not intended to be limited to such an example. Further, the catheter tube 208 may include at least a pair of catheter tube lumens and couple to a distal end of the molded hub 202. A distal end of each of the plurality of extension legs 212 may be coupled to a proximal end of the molded hub 202, where a fluid pathway is formed between the plurality of extension legs 212 and the catheter tube 208 through the pair of hub lumens.

Referring now to FIG. 2B, the catheter assembly 200 of FIG. 2A is shown where at least a portion of the adhesion bond has been broken such that at least a portion of the extension legs are separated. In some embodiments, the adhesion bond may be broken through the application of force pulling two or more of the extension legs 212 apart. The adhesion bond 214 can be placed at various lengths along the plurality of extension legs 212. For example, in some embodiments, the adhesion bond 214 can adhere the plurality of extension legs 212 together from the molded hub 202 to at least a majority of the length of the extension legs 214.

In some embodiments, the adhesion bond 214 detachably binds at least one extension leg to another, to allow separation of at least one of the plurality of extension legs 212. The adhesion bond 214 between the plurality of extension legs 212 can be detached via an external force. For example, in some embodiments, a medical professional can pull a Luer connector 204 connected to an extension leg 212, distally towards the molded hub 202, while securing the other Luer connectors 204 connected to the plurality of remaining extension legs 212 proximally (or otherwise away from the remaining extension legs 212). In some examples, one can detach at least one extension leg. In other examples, one can detach no more than one extension leg. In some examples, one can detach one or more extension legs to isolate a plurality of extension legs 212. For example, in some embodiments, the catheter assembly 200 can include a plurality of extension legs 212 including but not limited to at least three extension legs. In some embodiments, one can secure at least but not limited to one Luer connector 204 connected to one extension legs 212, pull distally towards the molded hub 202, while securing the additional two Luer connectors 204 connected to the two extension legs 204 to separate the plurality of extension legs 212 into two distinct plurality of extension legs 212. It should be understood that any number of extension legs that may be included in various embodiments of the catheter assembly 200 and the illustrations provided herein are not intended to be limiting.

### Catheter Assembly with Collar

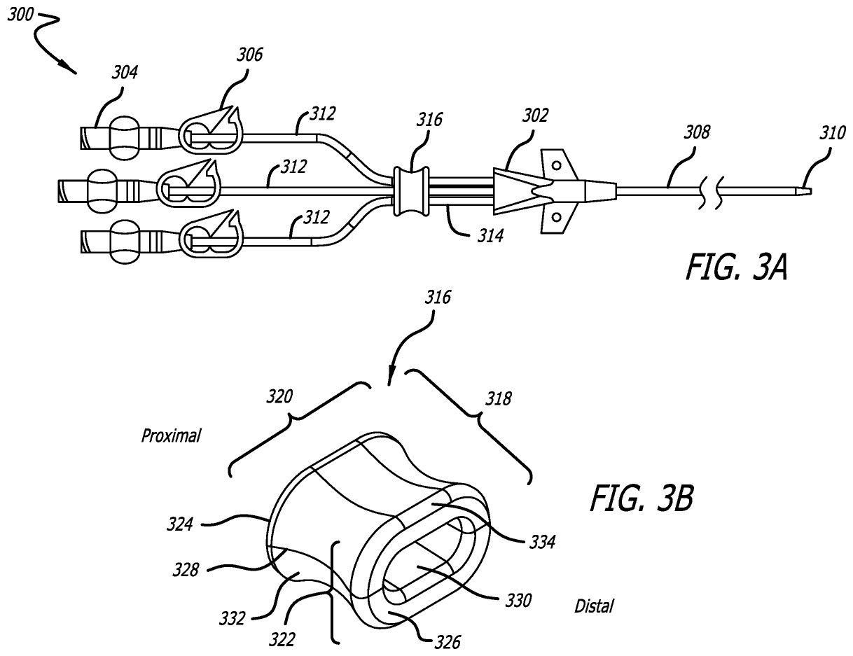

FIG. 3A illustrates a catheter assembly 300 including a molded hub 302 coupled to a catheter tube 308 containing a catheter tip 310. In some embodiments, the molded hub 302 is coupled to a plurality of extension legs 312 having clamps 306 and Luer connectors 304 disposed thereon. The catheter assembly 300 in some embodiments, optionally further includes a securing mechanism that includes an adhesion bond 314 between the plurality of extension legs 312 that secures the plurality of extension legs 312 together proximal the molded hub 302. The securing mechanism includes, in some embodiments, an adhesion bond 314, or a permanent slidable collar 316 or a combination thereof

FIG. 3B illustrates the permanent slidable collar 316 of FIG. 3A including a length 318, a width 320, a height 322, a proximal end, a proximal opening 324, a distal end, a distal opening 326, a longitudinal midpoint 332, a longitudinal endpoint 334, an outer surface 328, and an inner surface 330. In some embodiments, the length 318 can be within the range of 6-30 millimeters (mm). In some embodiments, the width 320 can be within the range of 6-30 mm. In different embodiments, the width 320 can be varied to accommodate a plurality of extension legs 312 according to the specific embodiment. The height 322 can be within the range of 6-30 mm. In some embodiments, the height 322 can be varied to accommodate a plurality of extension legs 312. The proximal opening 324 and the distal opening 326 can be, but is not limited to, in a shape configuration of an oval. In some embodiments, the proximal opening 324 and the distal opening 326 can include a shape configuration including circular, rectangular, square, polygonal including in some embodiments, hexagonal, heptagonal, octagonal, nonagonal or decagonal. The proximal opening 324 may include a shape configuration that is the same as the shape configuration of the distal opening 326. In some instances, the proximal opening 324 may include a shape configuration different from the shape configuration of the distal opening 326. In some embodiments, the longitudinal midpoint 332 is located in the middle of length 318 of the permanent slidable collar 316. As illustrated in FIG. 3B, the longitudinal endpoint 334 is located at the distal end of the permanent slidable collar 316.

In some embodiments, the outer surface 328 slopes from the proximal end in the direction of the inner surface 330 towards the longitudinal midpoint 332. In some embodiments, the outer surface 328 slopes from the longitudinal midpoint 332 away from the direction of the inner surface 330 towards the longitudinal endpoint 334 located at the distal end. In some embodiments, the permanent slidable collar 316 is formed having a continuous curve between the proximal end and the distal end. For example, the outer surface 328 slopes from the proximal end 324 in the direction of the inner surface 330 to a point at the longitudinal midpoint 332 then slopes away from the point at the longitudinal midpoint 332 away from the direction of the inner surface 330 towards the longitudinal endpoint 334 located at the distal end.

In some embodiments, the inner surface 330 extends along the length 318 to the longitudinal endpoint 334 located at the distal end. The inner surface 330 is configured to contact the outer surface of the plurality of extension legs 312. In some embodiments, the inner surface 330 is configured to create friction with an exterior of the extension legs 312. The friction prevents the permanent slidable collar 316 from moving distally without an external force applied upon it.

In an alternative embodiments, not shown in the figures, the outer surface 328 does not slope to the longitudinal midpoint 332 but extends along the length 318 to the longitudinal endpoint 334 located at the distal end. In other words, the outer surface 328 of the permanent slidable collar 316 is flat from the proximal end to the distal end.

In yet other embodiments, an underside of the collar 316 may include an adhesive disposed thereon to adhere to the patient's skin. In such an embodiment, the underside of the collar 316 may further include a removably liner that is initially disposed over the adhesive. The liner may include a pull tab enabling a medical professional to remove the liner and expose the adhesive. For example, the liner may be formed of paper and/or plastic.

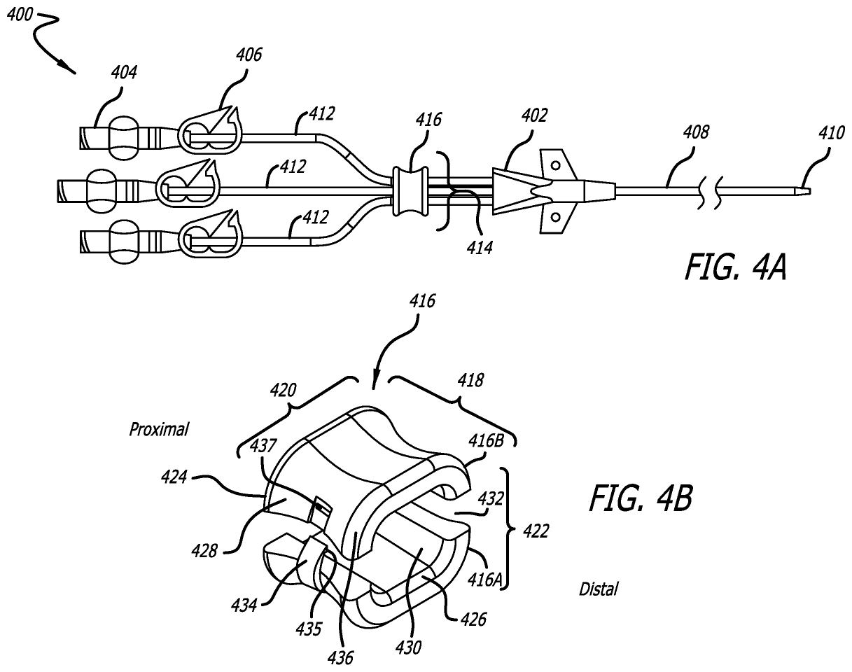

FIG. 4A illustrates a catheter assembly 400 including, in some embodiments, a molded hub 402 coupled to a catheter tube 408 containing a catheter tip 410. In some embodiments, the molded hub 402 is coupled to a plurality of extension legs 412 that contain clamps 406 and Luer connectors 404. The catheter assembly 400 in some embodiments, further includes a securing mechanism that includes an adhesion bond 414 between the plurality of extension legs 412 that secures the plurality of extension legs 412 together proximal the molded hub 402. The securing mechanism further includes, in some embodiments, an adhesion bond 414, or an attachable slidable collar 416 or a combination thereof.

FIG. 4B illustrates, in some embodiments, an attachable slidable collar 416 including a length 418, a width 420, a height 422, a proximal end, a proximal opening 424, a distal end, a distal opening 426 a longitudinal midpoint 432, a longitudinal endpoint 436, an outer surface 428 and an inner surface 430. The length 418 can be within a range 6 mm-30 mm. The width 420 can be within a range of 6-30 mm. The width 420 can be varied to accommodate a plurality of extension legs 412. In some embodiments, the height 422 can be within a range of 6-30 mm. In some embodiments, the height 422 can be varied to accommodate a plurality of extension legs 412 according to a specific embodiment. In some embodiments, the proximal opening 424 and the distal opening 426 can be but is not limited to include a shape configuration of an oval. In some embodiments, the proximal opening 424 and the distal opening 426 can include a shape configuration including circular, rectangular, square, polygonal including in some embodiments, hexagonal, heptagonal, octagonal, nonagonal or decagonal. In some embodiments, the proximal opening 424 may include a shape configuration that is the same as the shape configuration of the distal opening 426. In some embodiments, the proximal opening 424 may include a shape configuration different from the shape configuration of the distal opening 426. As illustrated in FIG. 4B, the longitudinal midpoint 432 is located in the middle of length 418. In some embodiments, the longitudinal endpoint 436 is located at the distal end.

In some embodiments, the attachable slidable collar 416 is formed having a continuous curve between the proximal end and the distal end. In some embodiments, the outer surface 428 slopes from the proximal end in the direction of the inner surface 430 towards the longitudinal midpoint 432. In some embodiments, the outer surface 428 slopes from the longitudinal midpoint 432 away from the direction of the inner surface 430 towards the longitudinal endpoint 436 located at the distal end of the attachable slidable collar. In some embodiments, the outer surface 428 may slope in one or more directions throughout the length 418. For instance, the shape of the outer surface 428 may be configured to slope in a first direction from either of the proximal or distal ends to a point throughout the length 418 (e.g., the midpoint 432).

As one non-limiting example, the outer surface 428 slopes from the proximal end in the direction of the inner surface 430 to a point at the longitudinal midpoint 432 then slopes away from the point at the longitudinal midpoint 432 away from the direction of the inner surface 430 towards the longitudinal endpoint 436 located at the distal end of the attachable slidable collar 416. In some embodiments, the outer surface 428 slopes from the proximal end in the direction of the inner surface 430 but does not slope to a point at the longitudinal midpoint 432 but instead in some embodiments, gradually slopes to the longitudinal midpoint 432 and gradually slopes away from the longitudinal midpoint 432 to the longitudinal endpoint 436 located at the distal end of the attachable slidable collar 416. In some embodiments, the outer surface 428 does not slope to the longitudinal midpoint 432 but extends along the length 418 to the longitudinal endpoint 436 located at the distal end of the attachable slidable collar 416. In other words, the outer surface of the attachable slidable collar 416 is flat from the proximal end to the distal end. In some embodiments, the inner surface 430 extends along the length 418 to the longitudinal endpoint 436 located at the distal end 426 of the attachable slidable collar 416. In some embodiments, the inner surface 430 can physically contact the outer surface of the plurality of extension legs 412. In some embodiments, the inner surface 430 is configured to create friction with an exterior of the extension legs 412 such that the friction prevents the permanent slidable collar 416 from moving distally without an external force applied upon it.

In some embodiments, the attachable slidable collar 416 can further be separated axially into two pieces 416A, 416B that are configured to attached together at the longitudinal midpoints 432 by, for example but not limited to, a clasping mechanism 434, which may include any of clasps, hinges, snaps, magnets, etc., or a combination thereof. In some embodiments, as shown in FIG. 4B, the clasping mechanism 434 disposed on piece 416A and may be a static clasp that includes a snap hook 435 at a distal end, where the snap hook 435 is configured to be received by a groove 437 disposed on piece 416B. In some embodiments, a first clasping mechanism 434 can reside at the longitudinal midpoint 432 on a first side while a second clasping mechanism 434 can reside at the longitudinal midpoint 432 on a second side (not shown).

In some embodiments, the slidable collar 416 may include an underside and have disposed thereon an adhesive and a liner as discussed above with respect to the slidable collar 316.

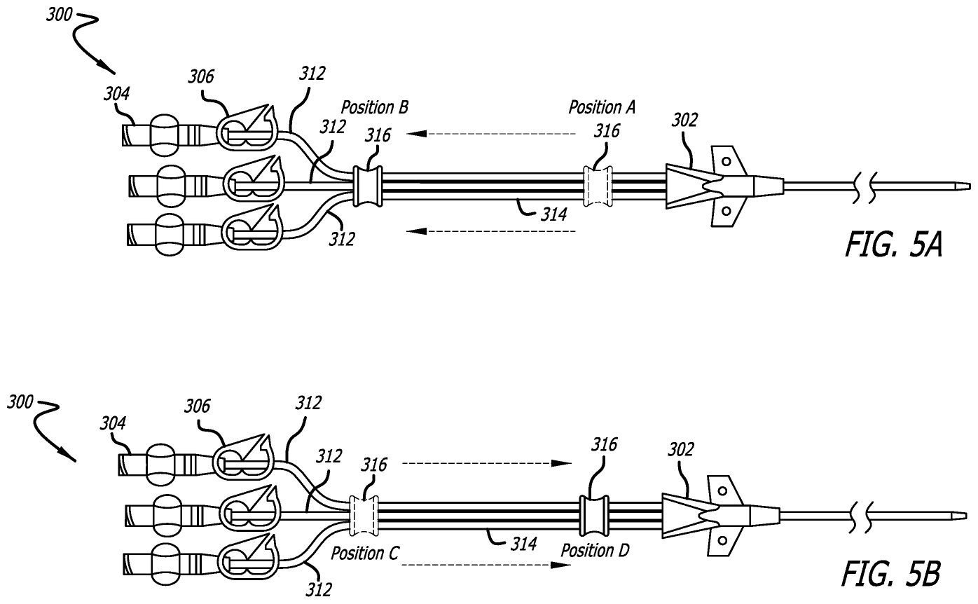

FIG. 5A provides an illustration that demonstrates how, in some embodiments, the permanent slidable collar 314 can be used to organize the plurality of extension legs 312 proximally the molded hub 302. In some embodiments, the plurality of extension legs 312 sit axially, contained within an inner cavity of the permanent collar 316 as shown in FIG. 3B. The permanent slidable collar 316 can move proximally from a position A to position B, due to the presence of an external force applied to the permanent slidable collar, which may have the benefit of gathering the plurality of extension legs 312, clamps 306 and Luer connectors 304 together.

FIG. 5B demonstrates how, in some embodiments, the permanent slidable collar 316 can be used to enable separation of the plurality of extension legs 312 from one another. In some embodiments, the permanent slidable collar 316 can move distally from position C to position D due to the presence of an external force applied to the permanent slidable collar, which, in the absence of an adhesive bond, such as the adhesive bond 314, may enable the plurality of extension legs 312, clamps 306 and the Luer connections 304 to separate. In some embodiments, position A may be equivalent to position D, and position B may be equivalent to position C; however, such is not required. The permanent slidable collar 316 can be moved proximally or distally along the plurality of extension legs 312. In some embodiments, the permanent slidable collar 316 can be detachable coupled to the molded hub 302. It should be noted that although the permanent slidable collar 316 is discussed above with respect to FIGS. 5A-5B, the same discussion applies equally for the attachable slidable collar 416.

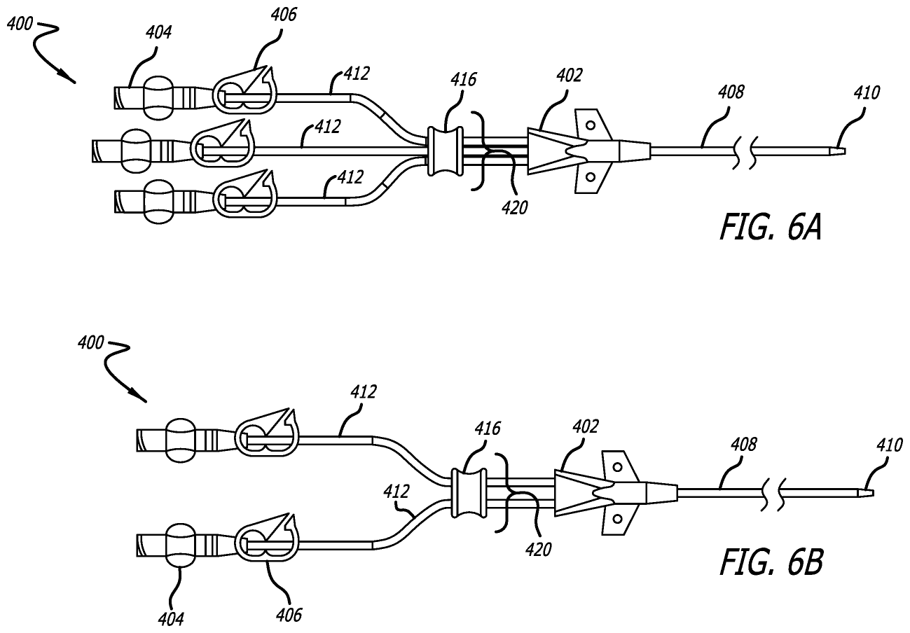

FIGS. 6A-6B illustrate alternative embodiments of slidable collars in accordance with some embodiments. In some embodiments, the width 420 of the attachable slidable collar 416 can be different depending on the plurality of extension legs 412. For example, in some embodiments, illustrated in FIG. 6A, the catheter assembly 400 has three extension legs 412. The width 420 of the attachable slidable collar 416 for the catheter assembly 400 with three extension legs 412 will be different in some embodiments than the width 420, illustrated in FIG. 6B, that has two extension legs 412. Further, the width 420 may be configured in accordance with the sizing of the extension legs (e.g., a diameter of each extension leg). It should be noted that although the attachable slidable collar 416 is discussed above with respect to FIGS. 6A-6B, the same discussion applies equally for the permanent slidable collar 316.

### Logical Flow of Manufacturing

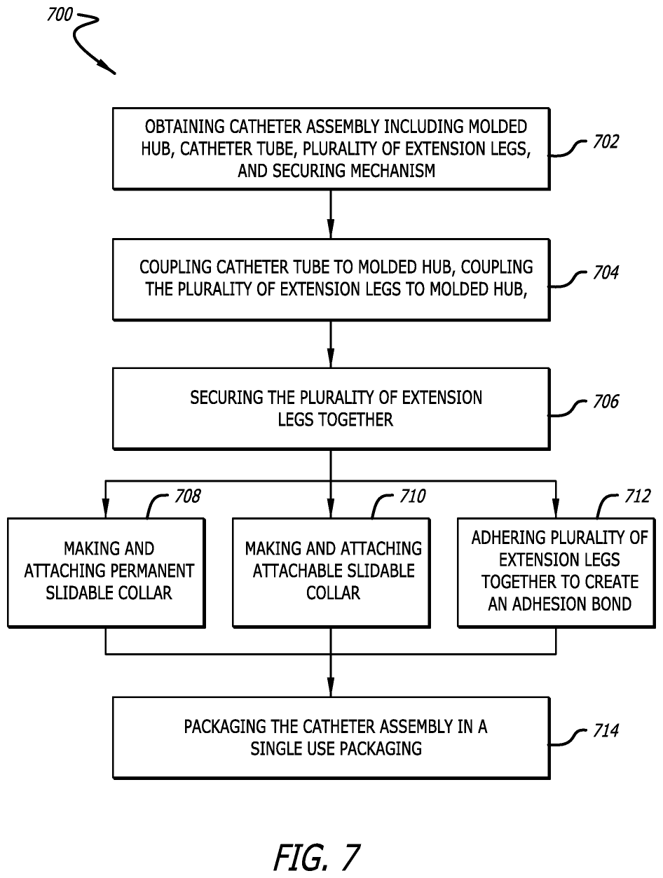

FIG. 7 is a flowchart illustrating a method 700 of making a catheter assembly for organizing extension legs, in some embodiments. In some embodiments, the method beings with obtaining a catheter assembly including a molded hub, a catheter tube, a plurality of extension legs and a securing mechanism (block 702). The method, in some embodiments, includes coupling the catheter tube to the molded hub and coupling the extension legs to the molded hub (block 704). The method 700 in some embodiments, further includes securing the plurality of extension legs together by, for example, adhering the plurality of extension legs together to create an adhesion bond (block 708), attaching the attachable slidable collar (block 710), attaching the permanent slidable collar (block 712) or a combination thereof In some embodiments, the adhering of the plurality of extension legs together to create an adhesion bond (block 712) can be by, but not limited to, a process including extrusion, co-extrusion or other commonly used manufacturing processes. In some embodiments, the plurality of extension legs can be adhered together post extrusion to form the adhesion bond such as through curing of the plurality of extension legs together through the application of heat to the plurality of extension legs. In some embodiments, the adhesion bond can include but is not limited to industrial glue, spray on adhesives, ultraviolet (UV) curing adhesives, etc.

In some embodiments, the curing process involves heating the extension legs to a temperature at which a plastic exterior coating begins to melt and the extension legs are then placed together. The cooling of the plastic exterior coating creates an adhesion bond between the extension legs. In some examples, the plurality of extension legs adhered together to create an adhesion bond may undergo a cooling step before the attaching the attachable slidable collar or attaching the permanent slidable collar is undergone (e.g., application of cooled air). In some examples, the plurality of extension legs may be configured with a barrier to prevent the distal movement of the attachable slidable collar or permanent slidable collar beyond a set point. This barrier may sit perpendicular to the direction of movement of the attachable slidable collar or permanent slidable collar and have a height with a range of 3-15 mm.

In some embodiments, the attaching the attachable slidable collar (block 710) or the attaching the permanent slidable collar (712), the attachable slidable collar or the permanent slidable collar may be configured to be initially bound to the molded hub so that an external force must be placed upon the attachable collar or permanent collar for any movement proximal the molded hub. In some embodiments, the attaching the attachable slidable collar (block 710) or the attaching the permanent slidable collar (712), the attachable slidable collar or the permanent slidable collar may be configured to be initially placed at the proximal end of the plurality of extension legs so that an external force must be placed upon the attachable slidable collar or permanent slidable collar for any movement distal the proximal end of the plurality of extension legs. In some embodiments, the attaching the attachable collar (block 710) or the attaching the permanent collar (712), the attachable slidable collar or permanent slidable collar may be initially attached anywhere along the plurality of extension legs.

In some embodiments, the method of making a catheter assembly includes making and attaching a permanent slidable collar (block 708) or making and attaching an attachable slidable collar (block 710). In some embodiments, the making of the permanent slidable collar (block 708) or the making of the attachable slidable collar (block 710) can be made from but not limited to acrylonitrile butadiene styrene, polyethylene, polycarbonate, nylon, silicon, polytetrafluoroethylene, acetal, polyphthalamine, polyphenylene sulfide, polyetheretherketone, high impact polystyrene, polypropylene, thermoplastic polyurethane, thermoplastic rubber, a combination thereof or other commonly used thermoplastics and thermoplastic derivatives used in the art.

In some embodiments, the method of making the permanent slidable collar 708 or making the attachable slidable collar (block 710) can further include an outer surface being constructed from a different material than the inner surface. For example, the inner surface can include acetal while the outer surface can include polyethylene. In some embodiments, the method of making the permanent slidable collar (block 708) or making the attachable slidable collar (block 710) can further include an outer surface being constructed from the same material as the inner surface. For example, the inner surface can include acetal while the outer surface can also include acetal. In some embodiments, the method of making the permanent slidable collar (block 708) or the method of making the attachable slidable collar (block 710) can be made by, but is not limited to the process of injection molding, 3D printing, urethane casting, thermoforming or other commonly used manufacturing techniques. In some embodiments, the attaching of the attachable collar may occur once the catheter assembly is in use by a patient. In some embodiments, the catheter assembly is packaged in a single use packaging once the plurality of extension legs are adhered together to create an adhesion bond (block 714).

### Exemplary Applications of Catheter Assembly Embodiments

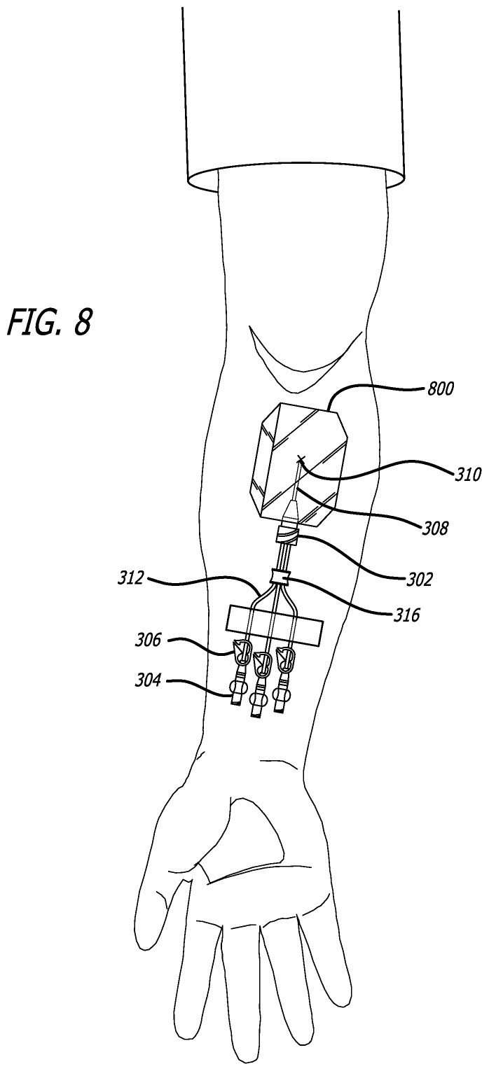

Referring to FIG. 8, an illustration of a catheter assembly including a slidable collar secured to a patient using a dressing is shown in accordance with some embodiments. FIG. 8 illustrates an exemplary catheter assembly, such as the catheter assembly 300 of FIG. 3, secured to a patient's arm with dressing 800. As shown, the dressing 800 may include an adhesive underside that adheres to the patient's arm. Procedurally, the catheter assembly 300 may be positioned on the patient's arm such that the catheter tip 310 is inserted into the patient at a percutaneous insertion site (marked with an ‘X’ in FIG. 8). Following insertion of the catheter tip 310, the catheter assembly 300 may be secured to the patient's arm by the placement of the dressing 800 directly over top of the catheter tube 308 and the insertion site ‘X’. In some instances, the dressing 800 may also be disposed over at least a portion of the molded hub 302. As shown, the slidable collar 316 may be disposed around the plurality of extension legs 312 proximal the molded hub 302 (where proximal is relative to the molded hub 102 such that the catheter tip 310 refers to a distal tip). The slidable collar 316 is shown to be operably disposed on plurality of extension legs 312 proximal the molded hub 302 and the dressing 800. Although the catheter assembly 300 is illustrated in FIG. 8, the catheter assembly 400 may also be utilized with the dressing 800. Further, the dressing 800 is merely an example illustration of one dressing; however, the disclosure is not intended to be so limited and instead any dressing known to those skilled the art may be utilized in place of the dressing 800 as illustrated.

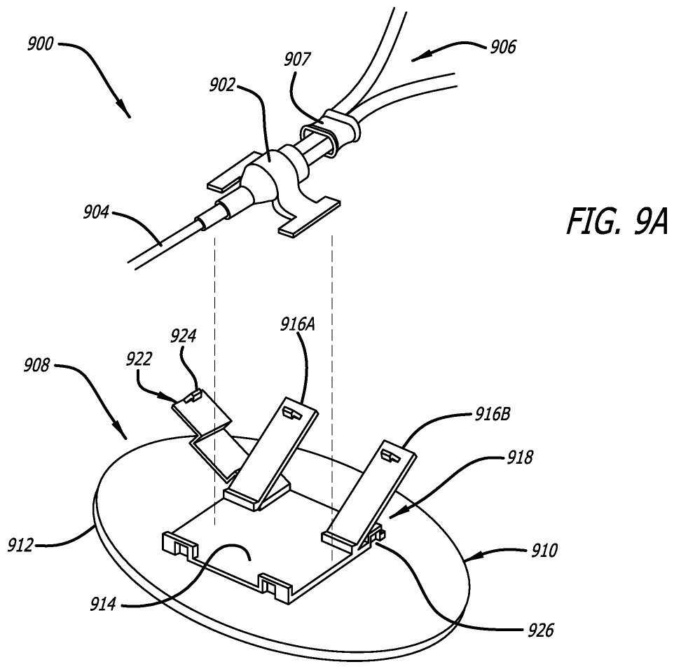

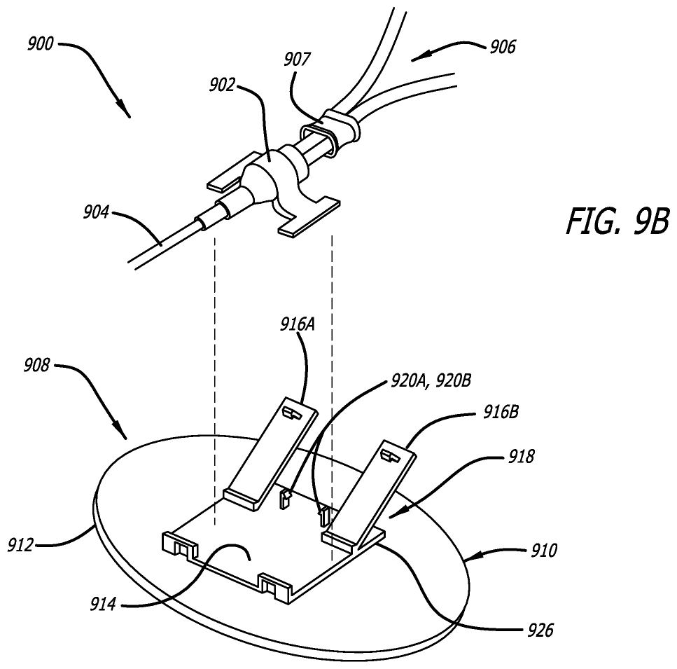

Referring to FIGS. 9A-9B, perspective views of two embodiments of anchoring systems are shown. Referring to FIG. 9A, the anchoring system 900 is constructed to couple with embodiments of the present invention. The anchoring system 900 comprises a securement device 908, which includes an anchor pad 910, upon which rests a retainer 914. A base of the retainer 914 supports a plurality posts and clips. Two covers 920A, 920B also extend from the retainer 914 and can be moved between open and closed positions to facilitate coupling with a medical device, such as the molded hub 902. The retainer 914 is configured to accept, to retain and to secure one or both catheter fittings (e.g., flanges extending laterally from the body of the hub 902) within the securement device 908.

As is illustrated, a plurality of extension legs 906 extend proximally from the molded hub 902 where a slidable collar 907 may be proximally disposed around a plurality of extension legs 906 the molded hub 902 (where proximal is relative to the molded hub 902 such that the catheter tube 904 extends distally from the molded hub 902). The slidable collar 907 may take the form and features of either the slidable collar 316 or 416 as discussed above.

When the hub 902 is located in a first inserted position, the fittings may be coupled with the base of the retainer 914, while the covers 916A, 916B extend in a closed position over at least a portion of the fittings. When in the closed position over the fittings, the covers 916A, 916B inhibit transverse motion of the fittings and consequently the hub 902.

In some embodiments, a secondary cover 922 may be disposed on the back portion 918 including a flange clip 924. The secondary cover 922 is configured to rotate between open and closed positions, where the flange clip 924 couples with the latch 926 to retain the secondary cover 922 in the closed position. A clinician may apply force to decouple the flange clip 924 and the latch 926 (which operates in the same manner as the covers 916A, 916B and the corresponding flange clips and latches).

The anchor pad 910 is securely attached to the skin of the patient by its lower surface 912, while the retainer 914 is securely attached to the upper surface of the anchor pad 910. The anchor pad 910 can comprise a laminate structure with an upper plastic (e.g., woven polyester), paper, or foam layer (e.g., closed-cell polyethylene foam) and a lower adhesive layer. The lower adhesive layer constitutes the lower surface 912 of the anchor pad. The lower surface 912 desirably is a medical-grade adhesive and can be either diaphoretic or nondiaphoretic, depending upon the particular application.

Referring to FIG. 9B, an exemplary embodiment of the anchoring system 900 having prongs 920A, 920B to secure the slidable collar 907 is shown in accordance with some embodiments. As an alternative securement device for the slidable collar 907 to the cover 922, the retainer 914 may include the prongs 920A, 920B. In embodiments in which the retainer 914 is configured to mate (couple) with the slidable collar 907, a back portion 918 of the retainer 914 may include prongs 920A, 920B that releasably grip the lateral sides of the slidable collar 907 when downward pressure is applied to the slidable collar 907. The slidable collar 907 may be removed from the grip of the prongs 920A, 920B by a clinician exerting a pulling force on the slidable collar 907 (or in some instances, on the plurality of extension legs 906).

While some particular embodiments have been disclosed herein, and while the particular embodiments have been disclosed in some detail, it is not the intention for the particular embodiments to limit the scope of the concepts provided herein. Additional adaptations and/or modifications can appear to those of ordinary skill in the art, and, in broader aspects, these adaptations and/or modifications are encompassed as well. Accordingly, departures may be made from the particular embodiments disclosed herein without departing from the scope of the concepts provided herein.

What is claimed is:

1. A catheter assembly for organizing a catheter system, comprising:
a molded hub including at least a pair of hub lumens;
a catheter tube coupled to a distal end of the molded hub, the catheter tube including at least a pair of catheter tube lumens;
a plurality of extension legs, each including a distal end coupled to a proximal end of the molded hub, wherein a fluid pathway is formed between the plurality of extension legs and the catheter tube through the pair of hub lumens; and
a securing mechanism configured to secure the plurality of extension legs together, wherein the securing mechanism is a slidable collar positioned over the plurality of extension legs.

2. The catheter assembly according to claim 1, wherein the slidable collar is a permanent collar comprising a length, a width and a height, having a proximal end with a proximal opening, a distal end with a distal opening, with an outer surface and an inner surface.

3. The catheter assembly according to claim 2, wherein the length is greater than or equal to the width, and the width is greater than or equal to the height.

4. The catheter assembly according to claim 2, wherein the outer surface slopes towards the inner surface at a longitudinal midpoint, and slopes away from the inner surface towards a longitudinal endpoint.

5. The catheter assembly according to claim 2, wherein the inner surface is configured to create friction with an exterior of the extension legs, wherein the friction resists movement of the slidable collar in an absence of an external force applied to the slidable collar.

6. The catheter assembly according to claim 2, wherein the proximal opening and the distal opening have a shape configuration of an oval.

7. A catheter assembly for organizing a catheter system comprising:
a molded hub including at least a pair of hub lumens;
a catheter tube coupled to a distal end of the molded hub, the catheter tube including at least a pair of catheter tube lumens;
a plurality of extension legs each including a proximal and a distal end, wherein the distal end of each extension leg is coupled to a proximal end of the molded hub, wherein a fluid pathway is formed between the plurality of extension legs and the catheter tube through the pair of hub lumens; and
a securing mechanism configured to secure the plurality of extension legs together, wherein the securing mechanism is selected from the group consisting of an adhesion bond between the plurality of extension legs, an attachable slidable collar, and a permanent slidable collar.

8. The catheter assembly according to claim 7, wherein the securing mechanism includes the adhesion bond between the plurality of extension legs.

9. The catheter assembly according to claim 7, wherein the securing mechanism is the attachable slidable collar.

10. The catheter assembly according to claim 9, wherein the attachable slidable collar is detachably coupled to the molded hub.

11. The catheter assembly according to claim 7, wherein the attachable slidable collar or permanent slidable is positioned over the plurality of extension legs.

12. The catheter assembly according to claim 7, wherein the securing mechanism is either of the attachable slidable collar or the permanent slidable collar, and wherein the securing mechanism comprises a length, a width, a height, having a proximal end with a proximal opening, a distal end with a distal opening, a longitudinal midpoint, a longitudinal endpoint and with an outer surface and an inner surface.

13. The catheter assembly according to claim 12, wherein the length is greater than or equal to the width, and the width is greater than or equal to the height.

14. The catheter assembly according to claim 12, wherein the outer surface slopes towards the inner surface at the longitudinal midpoint, then slopes away from the inner surface towards the longitudinal endpoint.

15. The catheter assembly according to claim 12, wherein the inner surface is configured to create friction with an exterior of the extension legs, wherein the friction resists movement of the attachable slidable collar in an absence of an external force applied to the attachable slidable collar.

16. The catheter assembly according to claim 12, wherein the proximal opening and the distal opening are oval shaped.

17. The catheter assembly according to claim 12, wherein the securing mechanism can be separated axially into two separate sections.

18. The catheter assembly according to claim 17, wherein the securing mechanism is joined at the longitudinal midpoints.

19. The catheter assembly according to claim 18, wherein the securing mechanism is joined together by a clasping mechanism.

20. The catheter assembly according to claim 19, wherein the clasping mechanism includes a clasp having a snap hook disposed on a first section and a groove on a second section, wherein the groove is configured to receive the snap hook.

21. The catheter assembly according to claim 18, wherein the securing mechanism is joined together by a first pair of magnets and a second pair of magnets, opposite the first pair of magnets.

22. A method of making a catheter assembly for organizing a catheter system comprising:
obtaining a molded hub including at least a pair of hub lumens, each of the hub lumens having an entry point adjacent or substantially adjacent to another entry point;
obtaining a catheter tube, the catheter tube including at least a pair of catheter tube lumens;
obtaining a plurality of extension legs;
obtaining a securing mechanism for securing the plurality of extension legs together, wherein the securing mechanism is selected from the group consisting of an adhesion bond, a permanent slidable collar, and an attachable slidable collar;
coupling the catheter tube to a distal end of the molded hub;
coupling a distal end of each of the plurality of extension legs to a proximal end the molded hub, wherein a fluid pathway is formed between the plurality of extension legs and the catheter tube through the pair of hub lumens;
securing the plurality of extension legs together using the securing mechanism; and
packaging the catheter assembly into a single use packaging.

23. The method of making a catheter assembly according to claim 22, wherein securing the plurality of extension legs together using the securing mechanism further comprises curing the plurality of extension legs together with heat to create the adhesion bond.

24. The method of making a catheter assembly according to claim 22, wherein securing the plurality of extension legs together using the securing mechanism further comprises coating the plurality of extension legs with an adhesive and curing with heat, pressure, or a combination thereof to create the adhesion bond.

25. The method of making a catheter assembly according to claim 22, wherein securing the plurality of extension legs using the securing mechanism further comprises attaching the permanent slidable collar.

26. The method of making a catheter assembly according to claim 22, wherein securing the plurality of extension legs using the securing mechanism further comprises attaching the attachable slidable collar.
